(19) United States
(12) Patent Application Publication
(10) Pub. No.: US 2012/0330208 A1
(43) Pub. Date: Dec. 27, 2012

(54) Bandage for prevention of skin erosion

(76) Inventor: William Bennett, Coronado, CA (US)

(21) Appl. No.: 13/529,242

(22) Filed: Jun. 21, 2012

Related U.S. Application Data
(60) Provisional application No. 61/499,769, filed on Jun. 22, 2011.

(51) Int. Cl. A61F 13/00

(52) U.S. Cl. 602/43; 602/48

(57) ABSTRACT

A cushioning bandage with absorbent properties, configured to permit easy positioning over a wound. The bandage comprises a highly lofted lamb's wool pad attached to variable lengths of elastic material. The bandage is configured for easy application to patients.

### CROSS-REFERENCES TO RELATED APPLICATIONS

This application claims the benefit of U.S. Provisional Application No. 61/499,769, filed 22 Jun. 2011, which is incorporated herein by reference.

### BACKGROUND OF INVENTION

1. Field of Invention

The inventive subject matter relates to a bandage that reduces or prevents skin erosion. The bandage is particularly valuable for covering implanted cardiac devices.

2. Background Art

Protection of wounds, such as ulcerous sores, is typically accomplished with the utilization of compression bandage systems. These typically encompass multiple layers. To alleviate improper compression application, a cushion, comprising a liquid or gel fill, such as silicone, can be used to completely cover the sore and the concomitant application of pressure against the pressure sore through the cushion (U.S. Patent Application no. U.S. 2010/0288291). Additionally, cushioned, multilayered cushioned bandages have been described that also contain an adhesive layer (U.S. Patent Application no. U.S. 2011/0077608).

For other needs, where absorbency is key, such as in sanitary towel, panty liner, and other bandage systems requiring highly absorption capacity, the use of high-loft material in combination with absorbent materials or fibers has been employed. For example, some stems and methods employ high loft material, typically a cellulose pulp, and overlaid with highly absorbent materials. Alternatively, the high loft material is interwoven with fibers with high absorption capacity, such as in U.S. patent application Ser. No. 6,235,966. In either case, in these systems, the primary concern is fluid absorption. Often, however, these systems can still cause considerable tissue erosion, such as wounds remaining following implant or cardiac surgery. Furthermore, many bandages systems are difficult to place over the wound, especially over extended periods of time.

### SUMMARY OF THE INVENTION

The current invention relates to a bandage and method of use that reduces or prevents skin erosion. The bandage is useful following any procedure where skin erosion is of concern, especially following implant surgery, such as cardiac implants, or in burn patients.

In a preferred embodiment, the bandage comprises a pad comprising lamb's wool or other, natural or synthetic highly lofted-material, with similar cushioning and absorbency properties. Attached at opposite ends of the pad is an elastic material. In one embodiment, the elastic material can be of pre-determined lengths to accommodate different size requirements. In an alternative embodiment, the elastic material on one or both sides of the pad can be adjustable so that the bandage can be adjusted to fit multiple sized individuals. Attached to the free ends of the elastic material is a means for connecting the ends of the elastic material together and also adjusting the fit of the bandage. The means can be any device capable of a secure connection, such as hook and loop fasteners.

### BRIEF DESCRIPTION OF DRAWINGS

FIG. 1. Diagram illustrating bandage components.

FIG. 2. Diagram of example of adjustment device for altering the length of the elastic material. Illustrated are alternative adjustment means, including a buckle, such as a double adjust side release buckle and wherein one end of the elastic material is brought in contact with the other end and secured by a loop and hook mechanism.

FIG. 3. Diagram, illustrating placement of bandage horizontally over torso. In this arrangement, it is preferred that the closure is positioned on the front of the torso.

FIG. 4. Diagram, illustrating potential methods of positioning of the bandage. Panel (A) illustrates bandage positioned across the chest and under the arms. Panel (B) and (C) illustrates a method of placement of the bandage, wherein the lofted portion such that the bandage is over the ipsilateral (B) or contra laterally (C) shoulder, with the other end under the opposite shoulder.

### DETAILED DESCRIPTION OF PREFERRED EMBODIMENTS

The invention relates to a bandage arrangement and method to reduce or prevent erosion of the skin. Use of the bandage arrangement is of particular value in medical situations where skin erosion, due to sensitivity of a wound area, and where longevity of wear and compliance of patients in protecting the wound area, are important medical considerations. Examples of use include implant wounds, such as following cardiac implant surgery. Additionally, not only is tissue erosion of paramount importance, but absorption of fluids away from the wound is important

Concomitant issues in current bandaging methods is the difficulty in easily applying and positioning the bandage to ensure adequate protection of the wound over extended periods of time. Protection of a wound in the long-term is particularly important in more ambulatory patients, such as patients who have undergone cardiac implant surgery, or other similar, longer standing wounds. In these situations, application of easy to apply wound protection systems that affords adequate protection against skin erosion and absorbency is paramount. Ease of application of the bandage is often important to also ensure patient compliance with maintaining wound protection.

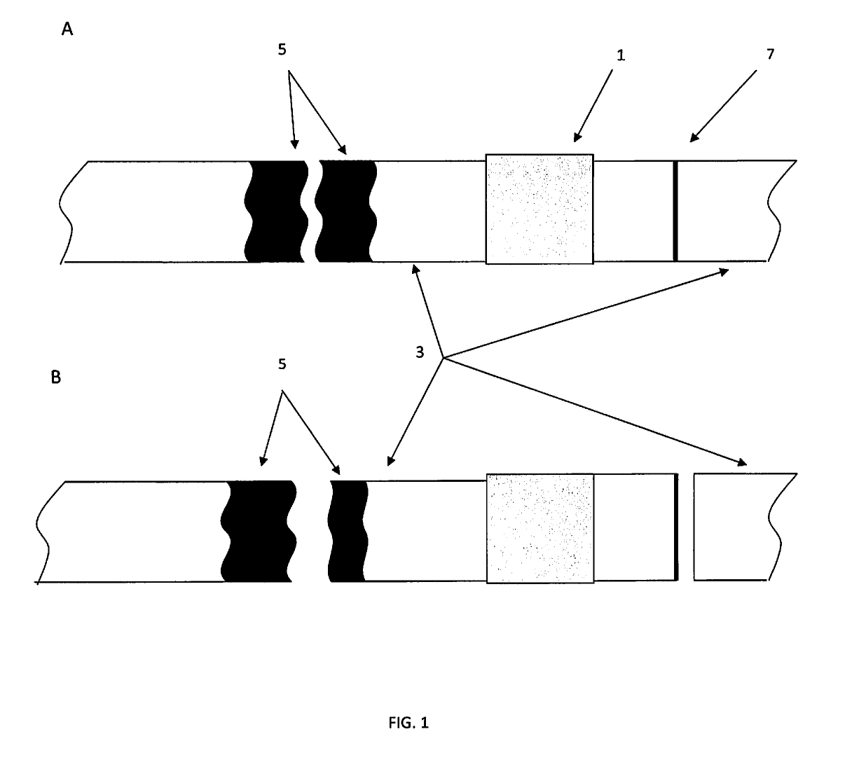

In order to address the need for a bandage that can adequately protect against skin erosion, provide adequate absorption capacity and be easy to apply over the wound for extended periods, a preferred embodiment is a bandage and method of its use, wherein the bandage comprises a non-woven or non-compressible material is envisioned as a preferred embodiment and is illustrated in FIG. 1. As shown in FIG. 1, the bandage comprises a pad made of a non-woven or non-compressible material (1), such as lofted-materials that is also absorbent. As used, herein, loft refers to the property of a material that contains low-density, material fibers with high air content. In this embodiment, the high loft material also has high absorption capacity, so as to not require other materials that would impeded materials wound cushioning ability.

In a preferred embodiment, the pad comprises the high-loft material lamb's wool. Lamb's wool alone, without other bandage layers, can provide adequate an adequate cushion for extremely sensitive wounds, such as that following cardiac implant surgery. Furthermore, in most cases, Lamb's wool is absorbent and can wick fluids away from the wound for wound drainage. In place of lamb's wool, other materials can be utilized in the pad, such as synthetic materials with similar cushioning and absorbency/fluid wicking characteristics as lamb's wool.

Also as illustrated in FIG. 1, the bandage also comprises, on opposite ends of the pad, either a single piece (FIG. 1 (A)) or two pieces (FIG. 1 (B)) of elastic material (3). The pad (1) is connected to the elastic material, as illustrated in FIG. 1. In one embodiment, the material can be a single piece of pre-cut lengths for easy positioning and application of the bandage on a defined size range of patients. In another embodiment, the length of the elastic material can be pre-adjusted to provide a proper fit on individuals of multiple sizes and ages.

For adjustable elastic material, after adjustment for the size of the elastic band to the appropriate size of the patient, the bandage can be secured on the patient by securing the free ends using the closures (5). Closures (5) are attached at each end of the elastic material, as illustrated in FIG. 1, which are used to finally secure and adjust the bandage securely to the patient. The closures (5) can be any means for finally adjusting the length of the elastic material and for firmly securing the bandage in place. As an example, a hook and loop fastener system can be used to quickly secure the ends of the elastic material.

The adjustable elastic material embodiment comprises an elastic material divided into two parts (3), as illustrated in FIG. 1 (B). In this embodiment, the combined lengths of the elastic material (3) are coarsely adjusted, to the appropriate size for the patient and connected using an adjustment loop (7) and securing means (9). Any method of adjustment can be used. An example is to thread the free end of one part of the elastic material through an adjustment loop (7) and then securing the free end with a securing means (9). An example of the placement of the adjustment loop (7) on the bandage is illustrated in FIG. 1.

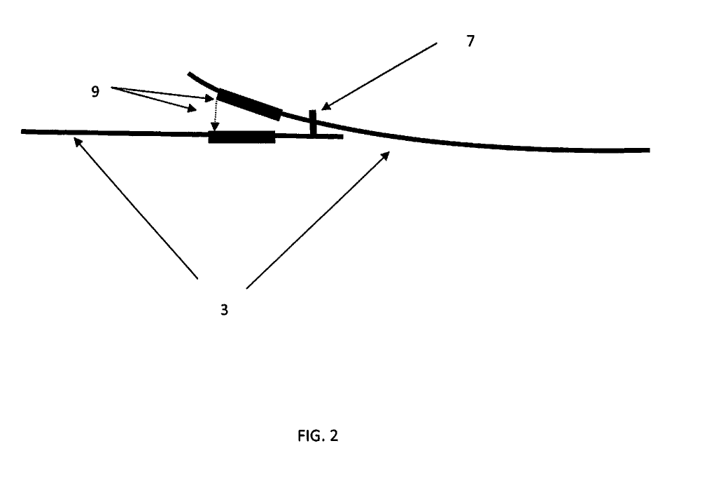

As an example, as illustrated in FIG. 2, adjustment of the elastic material can be by passing one end of the elastic material through an adjustment loop (7). The elastic material is then conveniently secured and held in place with a securing means (9). An example of a securing means (9) is a double adjust side release buckles. Alternatively, the elastic material can be adjusted by joining ends with a loop and hook mechanism, as illustrated in FIG. 2. Other types of buckles or devices that permit easy adjustment are also envisioned.

Regardless of adjustment mechanism or loop (7) that is utilized, the two elastic material pieces, as illustrated in FIG. 1 (B), are drawn through the loop (7), as illustrated in FIG. 2, until the bandage reaches a proper fit for a particular patient and the ends secured by securing means (9).

The convenient locations of the closures (5) and the adjustment loop (7) and securing means (9), in front of the patient, permits the bandage to be positioned and adjusted by a health-care worker or the patient, if necessary. The elastic aspects of the bandage and easy closure aspect makes positioning of the bandage easier and therefore more precise, maximizing the cushioning effective of the bandage and wound protection compliance by the patient.

The pad (1) is designed to maintain coverage of the wound area and protection from exposure to potentially erodible forces that could cause further tissue injury and infection. In alternate embodiments, the bandage (1) can be interlaced with anti-microbial materials, including antibiotics or anti-microbial metals, such as silver.

To reduce tissue erosion in a wound, a bandage, comprising a pad (1) comprising a high loft, high absorption material is applied to a patient. In a preferred embodiment, the pad (1) is wool or similar material with similar cushioning ability and ability to absorb and wick fluid.

In a preferred embodiment, the bandage pad (1) is firmly attached to an elastic material. Application of the bandage is by directly applying the pad (1) over the wound area. The pad (1) would be is then secured to the patient through closures (5) on each end of the elastic strip. The closures can be any means for securing the ends of the elastic strip, including hook and loop fasteners. The elastic strip of the bandage can be provided in pre-determined lengths designed for specific age or sizes of patients. In addition or alternatively, the elastic strips can contain an adjustment loop (7) and securing means (9) to lengthen or shorten the strip to firmly secure the bandage.

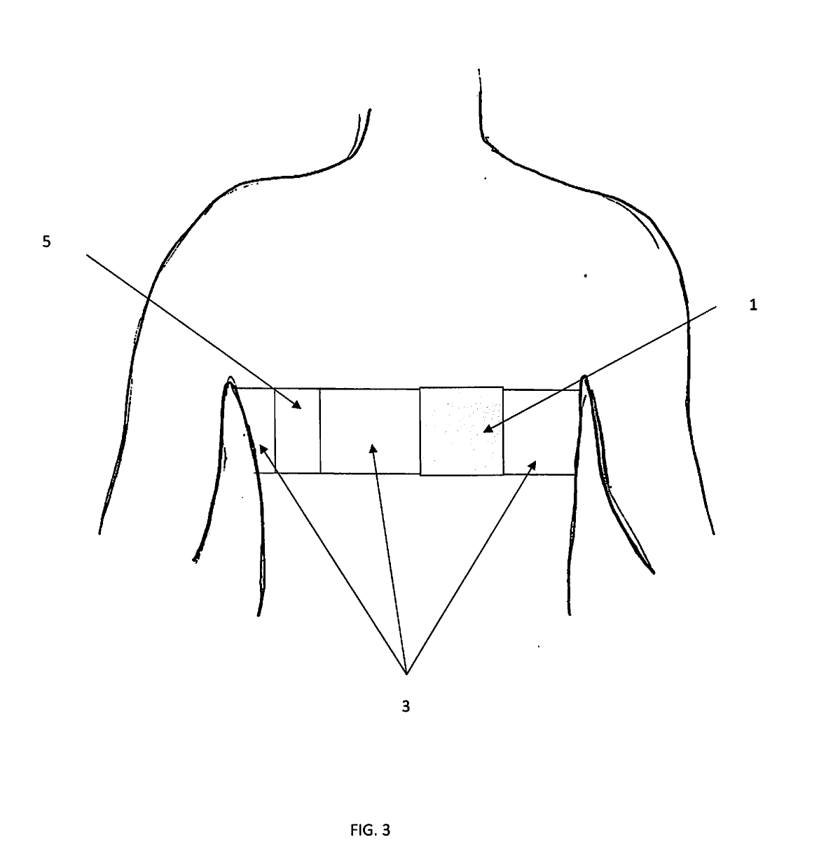

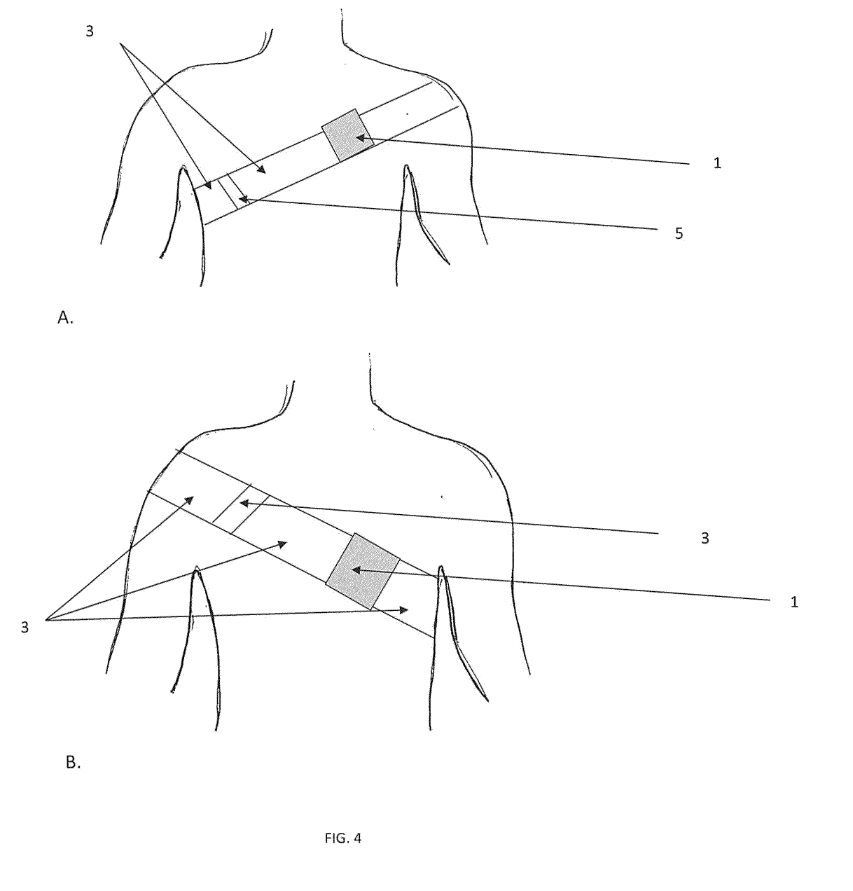

As an illustration, the bandage can be applied to a chest wound by placing the bandage horizontally across the torso with the elastic under the arms. The closures would, preferably, be situated on the front for easy access. This is illustrated in FIG. 3. Alternatively, as illustrated in FIG. 4, the bandage can be positioned across the chest and under the arms. The lofted portion of the bandage (i.e., pad (1)) is over the wound and the bandage is situated and position over the ipsilateral or contra lateral shoulder.

What is claimed is:

1. A bandage system comprising a pad containing a material that is high loft and absorbent connected to an elastic material with opposite free ends, which can be connected on its free ends by closures.

2. The bandage system of claim 1, wherein said high loft material is lamb's wool.

3. The bandage system of claim 1, wherein said closure is a hook and loop fastener.

4. The bandage system of claim 1, wherein said pad contains one or more antibiotic or antimicrobial agents.

5. The bandage system of claim 1, wherein the elastic material comprises two parts which are connected by a securing means to coarsely adjust the combined lengths of the connected two parts in order to fit multiple size patients.

6. The bandage system of claim 4, wherein said antimicrobial agent is an antimicrobial metal

7. The bandage system of claim 5, wherein said securing means is a buckle or a hook and loop fastener.

8. The bandage system of claim 6, wherein said antimicrobial metal is silver.

9. A method of cushioning a wound comprising the steps:
a. applying a pad to the wound containing a material that is high loft and absorbent to a wound;
b. securing said pad with two strips of elastic material each containing two ends, wherein said each strip of elastic material is attached on one of its ends to opposite sides of the pad and attaching the free ends of the elastic material strips by closure means.

10. The method of claim 7, wherein said pad contains lamb's wool.

11. The method of claim 7, wherein said closure means is a hook and loop closure.

12. The method of claim 7, wherein said pad and said closure means is secured on the front of the patient's torso.

13. The bandage system of claim 7, wherein the elastic material comprises two parts which are connected by a securing means to coarsely adjust the combined lengths of the connected two parts in order to fit multiple size patients.

14. The method of claim 7, wherein said pad contains one or more substances that are antibiotic or antimicrobial.

15. The method of claim 7, wherein said antimicrobial substance is an antimicrobial metal.

16. The bandage system of claim 13, wherein said securing means is a buckle or a hook and loop fastener.

17. The method of claim 15, wherein said antimicrobial metal is silver.
